(19) United States
(12) Patent Application Publication
(10) Pub. No.: US 2006/0276781 A1
(43) Pub. Date: Dec. 7, 2006

(54) Cannula cooling and positioning device

(76) Inventors: Daniel Warren van der Weide, Madison, WI (US); Susan Andrews-Winter, Fitchburg, WI (US); Fred T. Lee JR., Madison, WI (US)

Correspondence Address: Patula & Associates, P.C., 14th Floor, 116 S. Michigan Ave., Chicago, IL, 60603, US

(21) Appl. No.: 11/237,430

(22) Filed: Sep. 28, 2005

Related U.S. Application Data
(63) Continuation-in-part of application No. 10/834,802, filed on Apr. 29, 2004, now Pat. No. 7,101,369.
(60) Provisional application No. 60/679,722, filed on May 10, 2005.
(60) Provisional application No. 60/684,065, filed on May 24, 2005.
(60) Provisional application No. 60/690,370, filed on Jun. 14, 2005.
(60) Provisional application No. 60/702,393, filed on Jul. 25, 2005.
(60) Provisional application No. 60/707,797, filed on Aug. 12, 2005.
(60) Provisional application No. 60/710,276, filed on Aug. 22, 2005.
(60) Provisional application No. 60/710,815, filed on Aug. 24, 2005.

(51) Int. Cl. A61B 18/18

(52) U.S. Cl. 606/41; 606/33

(57) ABSTRACT

A cooling device comprises a thermally conductive material preferably having a large surface area, such as a plurality of fins. The cooling device clamps or slides onto an energy-introducing cannula and can exchange heat with the surrounding air, or with a coolant enclosed in a camber or shroud around the cooling device. The coolant can be circulated via a pump connected to the shroud. The device and/or shroud can be stabilized and positioned by a positioning cone or spacer. The cooling device and method reduces, minimizes or eliminates thermal effects at critical points along the cannula, while enabling the distal end of the cannula, at which treatment is occurring, to reach a temperature sufficient to kill tumor cells.

### CLAIM OF PRIORITY

This application is a Continuation-In-Part of co-pending U.S. Non-Provisional Patent Application entitled “Triaxial Antenna for Microwave Tissue Ablation” filed Apr. 29, 2004 and assigned U.S. application Ser. No. 10/834,802, the entire disclosure of which is hereby herein incorporated by reference.

This application further claims priority to U.S. Provisional Patent Applications entitled “Segmented Catheter for Tissue Ablation” filed May 10, 2005 and assigned U.S. Application Ser. No. 60/679,722; “Microwave Surgical Device” filed May 24, 2005 and assigned U.S. Application Ser. No. 60/684,065; “Microwave Tissue Resection Tool” filed Jun. 24, 2005 and assigned U.S. Application Ser. No. 60/690,370; “Cannula Cooling and Positioning Device” filed Jul. 25, 2005 and assigned U.S. Application Ser. No. 60/702,393; “Intralumenal Microwave Device” filed Aug. 12, 2005 and assigned U.S. Application Ser. No. 60/707,797; “Air-Core Microwave Ablation Antennas” filed Aug. 22, 2005 and assigned U.S. Application Ser. No. 60/710,276; and “Microwave Device for Vascular Ablation” filed Aug. 24, 2005 and assigned U.S. Application Ser. No. 60/710,815; the entire disclosures of each and all of these applications are hereby herein incorporated by reference.

### CROSS-REFERENCE TO RELATED APPLICATIONS

This application is related to co-pending U.S. Non-Provisional Patent Application entitled “Triaxial Antenna for Microwave Tissue Ablation” filed Apr. 29, 2004 and assigned U.S. application Ser. No. 10/834,802; and to U.S. Provisional Patent Applications entitled “Segmented Catheter for Tissue Ablation” filed May 10, 2005 and assigned U.S. Application Ser. No. 60/679,722; “Microwave Surgical Device” filed May 24, 2005 and assigned U.S. Application Ser. No. 60/684,065; “Microwave Tissue Resection Tool” filed Jun. 24, 2005 and assigned U.S. Application Ser. No. 60/690,370; “Cannula Cooling and Positioning Device” filed Jul. 25, 2005 and assigned U.S. Application Ser. No. 60/702,393; “Intralumenal Microwave Device” filed Aug. 12, 2005 and assigned U.S. Application Ser. No. 60/707,797; “Air-Core Microwave Ablation Antennas” filed Aug. 22, 2005 and assigned U.S. Application Ser. No. 60/710,276; and “Microwave Device for Vascular Ablation” filed Aug. 24, 2005 and assigned U.S. Application Ser. No. 60/710,815; the entire disclosures of each and all of these applications are hereby herein incorporated by reference.

### FIELD OF INVENTION

The present disclosure relates generally to medical devices, and in particular, to medical devices in the field of radiofrequency (RF) ablation and/or microwave ablation. Specifically, the present disclosure relates to a cooling and positioning device for a radiofrequency or microwave energy introduction cannula, and a method for cooling and positioning the same.

### BACKGROUND

Use of energy to ablate, resect or otherwise cause necrosis in diseased tissue has proven beneficial both to human and to animal health. Electrosurgery is a well-established technique to use electrical energy at DC or radiofrequencies (i.e. less than 500 kHz) to simultaneously cut tissue and to coagulate small blood vessels. Radiofrequency (RF) ablation of tumor tissue was developed from the basis of electrosurgery, and has been used with varied success to coagulate blood vessels while creating zones of necrosis sufficient to kill tumor tissue with sufficient margin.

Radiofrequency (RF) ablation is now being used for minimally invasive focal destruction of malignant tumors. Microwave ablation has many advantages over RF ablation, but has not been extensively applied clinically due to the large probe size (14 gauge) and relatively small zone of necrosis (1.6 cm in diameter) that is created by the only commercially available microwave ablation device, known under the trade name Microtaze, by Nippon Shoji, of Osaka, Japan, and having the following parameters: 2.450 MHz, 1.6 mm diameter probe, 70 W for 60 seconds. A discussion of this can be found in an article by Seki T, Wakabayashi M, Nakagawa T, et al. entitled “Ultrasonically guided percutaneous microwave coagulation therapy for small hepatocellular carcinoma.” (Cancer 1994; 74:817-825), which is herein incorporated by reference. This large probe size would not be compatible with percutaneous use in the chest, and would only be used with caution in the abdomen.

Additional problems, disadvantages and/or limitations associated with such known devices include patient burns caused by heat traveling from the distal end of the catheter to the proximal end during use of such known devices. Accordingly, there is a need for a device which overcomes the problems, disadvantages and limitations associated with these known devices and procedures. The present disclosure fulfills this need.

### SUMMARY

The present disclosure relates to a cooling device and method for a radiofrequency or microwave energy introduction cannula, providing for the effective delivery of radiofrequency (RF) and/or microwave power to achieve coagulative necrosis in primary or metastatic tumors while reducing or eliminating thermal effects at critical points along the structure. The device limits the conductive path for heat generated both at the ablation site and along the filter sections so that heat travel from the distal end of the catheter to the proximal end is minimized or eliminated. The device beneficially cools the critical portions of the cannula while enabling the distal end of the cannula, at which treatment is occurring, to reach a temperature sufficient to kill tumor cells.

The cooling device comprises a thermally conductive material preferably having a large surface area, such as a plurality of fins, providing for more efficient thermal exchange with its environment. The cooling device clamps or slides onto an energy-introducing tube or cannula which is connected with a connector to a source of radiofrequency or microwave energy. The device can exchange heat with the surrounding air, or be further enclosed in a shroud that has static coolant. The shroud can also be connected to a coolant recirculation pump by means of an inlet and outlet. The device and/or shroud can be stabilized and positioned by a positioning cone or stop.

Accordingly, it is one of the objects of the present disclosure to provide a method and device for cooling the exterior of an energy-introducing cannula or tube. Numerous other advantages and features of the disclosure will become readily apparent from the following detailed description, from the claims and from the accompanying drawings in which like numerals are employed to designate like parts throughout the same.

### BRIEF DESCRIPTION OF THE DRAWINGS

A fuller understanding of the foregoing may be had by reference to the accompanying drawings wherein:

FIG. 1 is a schematic cross-sectional view of the cooling device of the preferred embodiment of the present disclosure.

FIG. 2 is a schematic diagram of the cooling device of the preferred embodiment of the present disclosure.

FIG. 3 is a schematic cross-sectional view of an alternate embodiment of the cooling device of the present disclosure.

FIG. 4 is a schematic cross-sectional view of another alternate embodiment of the cooling device of the present disclosure.

### DESCRIPTION OF DISCLOSED EMBODIMENT(S)

While the invention is susceptible of embodiment in many different forms, there is shown in the drawings and will be described herein in detail one or more embodiments of the present disclosure. It should be understood, however, that the present disclosure is to be considered an exemplification of the principles of the invention, and the embodiment(s) illustrated is/are not intended to limit the spirit and scope of the invention and/or the claims herein.

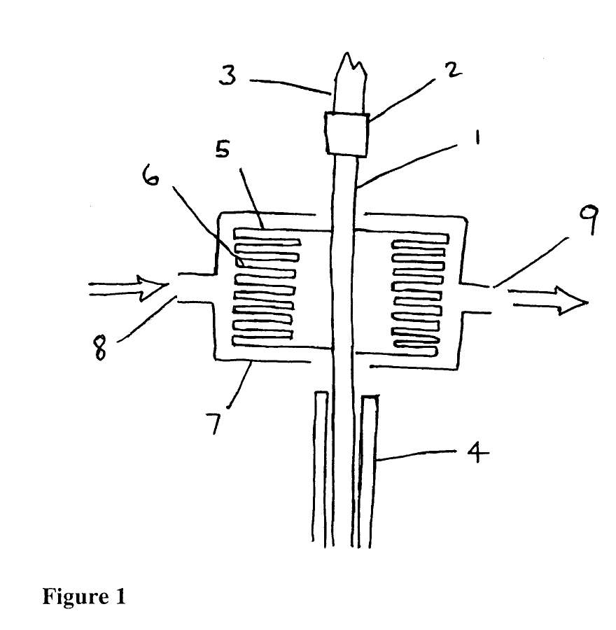

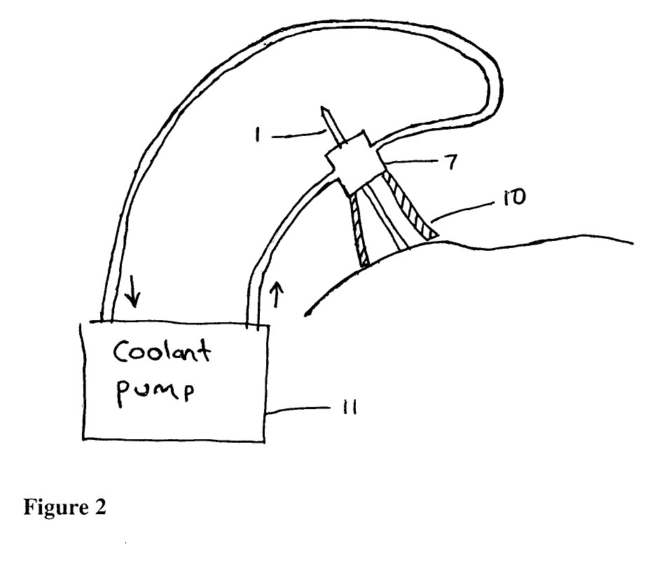

FIGS. 1 and 2 illustrate a cooling device and method for a radiofrequency or microwave energy introduction cannula (1), providing for the effective delivery of radiofrequency (RF) and/or microwave power to achieve coagulative necrosis in metastatic tumors while reducing or eliminating thermal effects at critical points along the structure. The cannula (1) or tube is a probe small enough to be used safely virtually anywhere in the neck, chest, abdomen, and pelvis, and be guided by computerized tomography (CT), MRI, or ultrasonic imaging.

The distal portion of the cannula (1) may be resonant at a frequency of interest (a drive frequency), typically one falling in the Industrial, Scientific, and Medical (ISM) band, covering approximately 800 MHz to 6 GHz, where efficient sources of ablative power (e.g. >5 watts output) are available, although the cannula may also be excited at RF. The resonant antenna structure is comprised of one or more resonant sections of coaxial, triaxial or multi-axial transmission line, which can form a multi-section filter that passes the drive frequency with essentially no loss, but is incapable of efficiently conducting power at other frequencies. At the distal end, the interior conductor(s) extend from the more exterior conductors in a telescoping fashion at lengths that are resonant at the drive frequency when the catheter is inserted into the tissue to be ablated.

The device limits the conductive path for heat generated both at the ablation site and along the filter sections so that heat travel from the distal end of the catheter to the proximal end is minimized or eliminated. By segmenting the catheter into one or more divisions, each division itself being a resonant length, electric-field coupling between adjacent segments can be preserved while interrupting the path for thermal conduction. The segmented catheter is reinforced with non-conducting materials in the gaps between segments, as well as (optionally) with a stiff inner conductor wire, thus preserving mechanical stability needed for insertion.

The preferred embodiment of the cannula is a resonant coaxial, triaxial or multiaxial structure whose resonant lengths are set 2.45 GHz in the tissue of interest; the catheter can be readily impedance-matched to the tissue by adjusting the length of its coaxial center conductor with respect to its shield, which itself can fit inside one or more introducer needles of total diameter less than 12 gauge. Impedance matching to tissue is done iteratively, using a RF or microwave network analyzer to achieve a low power reflection coefficient. Because its microwave reflection coefficient is low (typically −40 dB or better), the catheter can deliver˜100 W of power to the tissue with minimal heating of the catheter shaft, creating focal zones of coagulative necrosis >3 cm in diameter in fresh bovine liver. To achieve high power economically, a magnetron power supply is used, with a waveguide-to-coaxial transition and a dual-directional coupler to measure incident and reflected power during use.

To achieve larger zones of necrosis, multiple triaxial probes can be deployed using either a switch or power splitter to distribute the RF or microwave power.

With reference to the drawings, an example of the preferred embodiment of the cooling device of the present disclosure is shown in FIG. 1. As shown in FIG. 1, the cooling device clamps or slides onto an energy-introducing tube or cannula (1) which is connected with a connector (2) to a source of radiofrequency or microwave energy (3). The cannula (1) can be inserted into an introducer needle (4). The device (5) is made of a thermally conductive material such as copper or aluminum, though preferably the same material as that of the cannula. It is further given a larger surface area for more efficient thermal exchange with its environment by using fins (6). The device can exchange heat with the surrounding air, or be further enclosed in a shroud (7) that has static coolant (including but not limited to ice, dry ice, or an endothermic chemical reaction). The shroud (7) can also be connected to a coolant recirculation pump by means of an inlet (8) and outlet (9). Such coolant can be Freon, water, argon, or other suitable fluid.

An advantage of the cooling device is that it is universally adaptable to all energy introduction cannulas, and that it does not require a hollow cannula, or flow of coolant through the cannula. The external cooling of the cannula eliminates the need to increase the probe size to allow for internal cooling. Internally cooled systems require an in and out channel which necessitates a bigger probe.

A further object of the present disclosure is that the energy-reflective junctions such as the connector (2) are beneficially cooled by proximity to the device (5). A further object of the present disclosure is that the introduction of the cannula and introducer to skin is a point that is also close to the device, and is a critical point for avoiding patient burns. Thus this device beneficially cools the critical portions of the cannula while enabling the distal end of the cannula, at which treatment is occurring, to reach a temperature sufficient to kill tumor cells.

As shown in FIG. 2, the device (5) attached to the cannula tube (1) can be enclosed in a shroud (7) which is further stabilized and positioned by a positioning cone (10). This maintains optimal placement of the cannula and helps to monitor whether it has been moved during the procedure, or during patient positioning. The shroud is connected to a recirculating cooling pump (11) for maximum controlled cooling.

One or more thermocouples can be operatively associated with the cannula to sense the temperature at critical points along the cannula. The output of these thermocouples can be used to control the coolant pump and regulate the flow of coolant to ensure safe thermal operation.

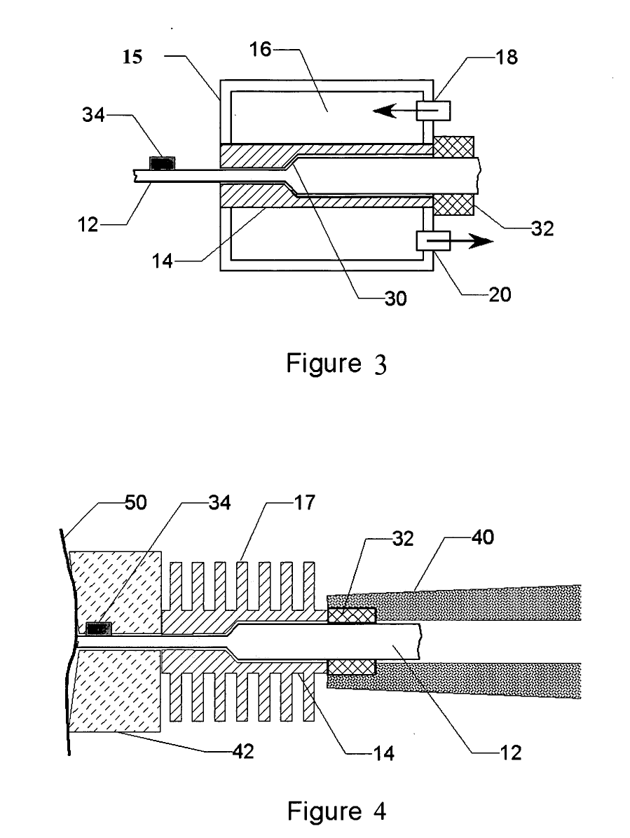

Referring now to the embodiments of FIGS. 3 and 4, the cooling device generally comprises a sheath for cooling the cannula. The thermally conductive core of the sheath may fully or only partially enclose the circumference of the cannula, but has a cooler mechanism in thermal contact with the core. The cooler mechanism is realized with one or more well known techniques, including fluidic heat exchange, the Peltier effect, cold solids, Joule-Thompson effect, or endothermic chemical reactions. The core may be shaped both to enhance thermal contact with the cannula and to provide a stop to determine the proper insertion depth for the cannula within the core. The sheath may be simply fixed or clamped onto the cannula, or the sheath may also serve as a handle to help position and insert the cannula. The sheath may also have a thread, clamp, clip, friction fit or expansion joint to hold the cannula in place, and the sheath may have a spacer to limit the insertion depth of the cannula.

Specifically, FIG. 3 illustrates a schematic cross-section of a sheath for cooling a cannula, and shows the cannula inserted into and in contact with the thermally conductive hollow core, which uses a fluidic heat exchanger whose fluid flow into and out of the exchanger is indicated by the arrows. The heat exchanger chamber 15 may also serve as a handle. In this embodiment, the housing 15 for the fluidic heat exchanger 16 also serves as a handle for holding and manipulating the cannula 12. Fluidic exchange is accomplished by inlet of cooling fluid 18, circulation of the fluid through the heat exchanger 16, which cools the hollow core 14 that fully encircles the cannula. Waste heat from the cannula travels with the cooling fluid through outlet 20. Cannula temperature is monitored by one or more temperature sensors 34, such as thermocouples, in thermal contact with the cannula.

At the proximal end of the cannula, a tapered transition or stop 30 both enhances thermal contact to the core 14 and provides a limit for insertion of the cannula. This tapered transition 30 may conjoin the cannula to a source of energy (such as microwave energy) to be introduced through the cannula, such as a coaxial, triaxial, or quadraxial cable or other conductor. Preferably, a clamp, clip, or thread 32 restrains the cannula once it is in place.

Again, the cooler mechanism may be realized with one or more well known techniques, including fluidic heat exchange, the Peltier effect, cold solids, Joule-Thompson effect, pellets of water ice or dry ice, or endothermic chemical reactions.

FIG. 4 is a schematic cross-section of a sheath for cooling a cannula, and shows an alternative fluidic heat exchanger, which is cooled by ambient air by means of cooling fins 17. Also shown is a hollow handle 40 attached to the heat exchanger and a clamp 32 embedded in the handle. A spacer 42 is shown to limit the insertion depth of the cannula through the skin 50. As shown in FIG. 4, the sheath may be simply fixed or clamped onto the cannula with a handle attached to the sheath. The sheath may also have a thread, clamp, clip, friction fit or expansion joint 32 to hold the cannula in place, and the sheath may have a spacer 34 to limit the insertion depth of the cannula into the skin 50.

It is to be understood that the embodiment(s) herein described is/are merely illustrative of the principles of the present invention. Various modifications may be made by those skilled in the art without departing from the spirit or scope of the claims which follow.

1. A device for cooling a radiofrequency or microwave energy introduction cannula, comprising:
a thermally conductive material element, the thermally conductive material element adapted to externally engage a portion of the treatment cannula;
wherein the thermally conductive material element exchanges heat with its surrounding.

2. The device of claim 1, further comprising a shroud surrounding the thermally conductive material element.

3. The device of claim 2, wherein the shroud includes a coolant.

4. A method for cooling the exterior of a radiofrequency or microwave energy introduction cannula, comprising the steps of:
positioning a thermally conductive material element on an external portion of the cannula; and
cooling the cannula via heat exchange from the thermally conductive material element and its surrounding.

5. A device for cooling a radiofrequency or microwave energy introduction cannula, comprising:
a thermally conductive hollow core adapted to receive and at least partially surround an exterior portion of the cannula, and
a cooler mechanism in thermal communication with the core.

6. The device of claim 5 wherein the cooler mechanism is a fluidic heat exchanger.

7. The device of claim 5 where the cooler mechanism is a Peltier-effect or Joule-Thompson cooler.

8. The device of claim 5 where the cooler mechanism is a cold solid.

9. The device of claim 5 where the cooler mechanism is an endothermic chemical reaction.

10. The device of claim 5 where the core includes a stop adapted to position the cannula within the core.

11. The device of claim 5 further comprising a handle attached to the core.

12. The device of claim 5 further comprising a temperature sensor in thermal contact with the cannula for controlling the cooler mechanism.

13. The device of claim 5 further comprising a clamp, clip, thread, friction fit, adhesive or expansion joint to hold the cannula relative to the core.

14. The device of claim 5 further comprising a spacer proximate the core to limit an insertion depth of the cannula into a treatment area.

15. The device of claim 14, wherein the spacer is a positioning cone.

16. The device of claim 1, wherein the thermally conductive material element includes a plurality of fins.

17. The method of claim 4, wherein the thermally conductive material element includes a plurality of fins.

18. The device of claim 5, wherein the thermally conductive hollow core includes a plurality of fins.

19. The device of claim 1, wherein the cannula comprises a segmented catheter for tissue ablation comprising one or more resonant sections of co-axial, triaxial or multi-axial transmission line.

20. The device of claim 5, wherein the cannula comprises a segmented catheter for tissue ablation comprising one or more resonant sections of co-axial, triaxial or multi-axial transmission line.
